(19) United States
(12) Patent Application Publication
(10) Pub. No.: US 2021/0398661 A1
(43) Pub. Date: Dec. 23, 2021

(54) MATCHING ASSISTANCE DEVICE, MATCHING ASSISTANCE METHOD, AND NON-TRANSIENT RECORDING MEDIUM ON WHICH MATCHING ASSISTANCE PROGRAM IS RECORDED

(71) Applicant: OMRON HEALTHCARE Co., Ltd., Kyoto (JP)

(72) Inventors: Tetsuya SATO, Kyoto (JP); Kosuke INOUE, Kyoto (JP); Mika KIJIMUTA, Kyoto (JP)

(21) Appl. No.: 17/446,687

(22) Filed: Sep. 1, 2021

Related U.S. Application Data
(63) Continuation of application No. PCT/JP2020/006084, filed on Feb. 17, 2020.

(30) Foreign Application Priority Data
Mar. 5, 2019 (JP) 2019-039689

(51) Int. Cl. G16H 40/20

(52) U.S. Cl. CPC G16H 40/20

(57) ABSTRACT

A matching assistance device, a matching assistance method, and a non-transient recording medium on which a matching assistance program is recorded, which assist matching between a patient and a practitioner who can perform a suitable treatment for the patient. When a search request for a practitioner matching the desire of a patient is received from a patient-side terminal used by the patient, a control unit of a server retrieves the practitioner information, from a practitioner information DB, that matches the patient information of the patient and returns information for displaying the search result to the patient-side terminal. When a display request for treatment history information associated with the patient information is received from a practitioner-side terminal used by a practitioner whose practitioner information is registered in the practitioner information DB, the control unit transmits information for displaying the treatment history information to the practitioner-side terminal.

### CROSS-REFERENCE TO RELATED APPLICATIONS

This application is the U.S. national stage application filed pursuant to 35 U.S.C. 365(c) and 120 as a continuation of International Patent Application No. PCT/JP2020/006084, filed Feb. 17, 2020, which application claims priority to Japanese Patent Application No. 2019-039689, filed Mar. 5, 2019, which applications are incorporated herein by reference in their entireties.

### TECHNICAL FIELD

The present invention relates to a matching assistance device, a matching assistance method, and a non-transient recording medium on which a matching assistance program is recorded.

### BACKGROUND ART

With the spread of personal computers (PCs) and the Internet, services for mediating between users who request predetermined information and companies are being used. Such services include job placement services and marriage partner introduction services. When using these services, a user registers personal information, such as age and educational background, and a category of interest with a service providing company, and matching processing is performed using these elements as criteria to search for a user or a company that matches the criteria.

For example, Patent Document 1 describes a job-seeking assistance system that mediates between job seekers and employers, and describes a technology that allows employers to search for job seekers that match the recruitment requirements of the employers based on personal information registered by the job seekers, such as age, educational background, and work history.

### CITATION LIST

### Patent Literature

Patent Document 1: JP 2006-72462 A

### SUMMARY OF INVENTION

### Technical Problem

Technology for matching between service providers and service requesters is conventionally known. However, there is no technology that assists matching between a practitioner, such as a physical therapist, bone setter, acupuncturist, moxibustion therapist, occupational therapist, or osteopath, who performs a treatment to care for and improve a person's physical condition, and a patient wishing to receive such treatment.

For example, when a patient has a physical problem, the patient will visit an orthopedic or osteopathic clinic for treatment. However, when visiting a hospital in such a case, patients are often not able to appoint a practitioner, which can cause the patient to be dissatisfied with the treatment. Also, the practitioner may not always be able to treat a patient who can be treated using his/her own area of expertise, which can cause dissatisfaction for the practitioner. In addition, patients can only receive treatment during the clinic's business hours, which can lead to waiting times and the inability to make time for visits. Practitioners are also only allowed to perform treatments during the clinic's business hours, which limits their ability to improve their own skills.

In order to solve these problems, a system for achieving optimal matching between a patient and a practitioner is considered effective. This system searches for a practitioner that satisfies various conditions (treatment time slot, specialty treatment site, years of experience, gender, etc.) desired by the patient from among a large number of pre-registered practitioners, and allows the patient to be treated by the searched practitioner. However, in providing services such as treatment to care for or improve the physical condition, suitable treatment for the patient may not be performed, even when the various predetermined requirements described above match the patient's wishes.

The purpose of the present invention is to provide a matching assistance device, a matching assistance method, and a matching assistance program that assistance matching between a patient and a practitioner who can perform a suitable treatment for the patient.

### Solution to Problem

(1)

There is provided a matching assistance device that assists matching between a practitioner who performs a treatment and a patient who desires a treatment, the device including: a practitioner information database in which practitioner information for each of a plurality of the practitioners is registered; a patient information database in which patient information of the patient is registered; a treatment history information database in which treatment history information associated with the practitioner information and the patient information of the patient who has received a treatment from a practitioner whose practitioner information is registered in the practitioner information database is registered; a matching processing unit configured to, when a search request for one of the practitioners matching the desire of the patient is received from a patient-side terminal used by the patient, retrieve the practitioner information, from the practitioner information database, that matches the patient information of the patient and return information for displaying the search result to the patient-side terminal; a first transmission unit configured to, when a display request for the treatment history information associated with the patient information is received from a practitioner-side terminal used by the practitioner whose practitioner information is registered in the practitioner information database, transmit information for displaying the treatment history information to the practitioner-side terminal; a second transmission unit configured to, when a display request for the treatment history information associated with the patient information of the patient is received from the patient-side terminal used by the patient, transmit information for displaying the treatment history information to the patient-side terminal; and an information addition unit configured to, when a request to add advice information regarding treatment details included in the treatment history information is received from the practitioner-side terminal, add advice information received from the practitioner-side terminal and information of the practitioner who uses the practitioner-side terminal to the treatment history information, and register the treatment history information in the treatment history information database, wherein the information of the practitioner who added the advice information included in the treatment history information is displayed in a format that does not identify the practitioner at the practitioner-side terminal, and is displayed in a format that identifies the practitioner at the patient-side terminal.

According to (1), for example, a practitioner who receives a request for treatment from a patient based on the result of the matching processing can request the display of the treatment history information associated with the patient information of the patient from the practitioner-side terminal to confirm past treatment history information of the patient at the practitioner-side terminal. This allows practitioners to increase the number of factors by which to determine whether they can provide a suitable treatment for a patient based on the patient's past treatment history, thereby enabling practitioners to encounter patients who are more suited to them. In addition, by disclosing their own treatment history information, patients can expect to be selected by a practitioner who can provide a suitable treatment for them, and can encounter a practitioner who is suited to them.

Additionally, other practitioners who have never treated the patient can add advice information to the treatment history information associated with the patient. This allows that patient to view the advice information included in his/her treatment history information and consider using the other practitioner who gave that advice. Further, by requesting treatment from the other practitioner, the patient can expect to improve his/her symptom. In this way, the advice makes it possible to increase the number of determination factors when selecting a practitioner, and more optimal matching between the patient and the practitioner can be expected. Additionally, for practitioners, the addition of advice allows them to make effective use of their own knowledge and skills, and to promote their own knowledge. As a result, it is possible to achieve matching that is beneficial to both the patient and the practitioner.

Further, the patient can obtain a sense of security by knowing the name and other details of the practitioner who gave the advice. On the other hand, a practitioner who has given advice to another practitioner can prevent other practitioners from knowing that fact, thereby preventing problems between practitioners.

(2)

There is provided the matching assistance device according to the above (1), wherein when a request to add, to the treatment history information to which the advice information has been added, information of an opinion regarding the advice is received from the practitioner-side terminal used by the practitioner identified by the practitioner information associated with the treatment history information, the information addition unit adds the information of the opinion received from the practitioner-side terminal to and registers the treatment history information in the treatment history information database.

According to (2), the practitioner who has treated the patient can add an opinion comment regarding advice by another practitioner who has never treated the patient, to the treatment history information associated with the patient. This allows that patient, for example, to make a decision to request another treatment from the practitioner who made the opinion comment when the opinion comment is reasonable, or to make a decision to request treatment from the practitioner who gave the advice when the opinion comment is unreasonable. In this way, opinion comments can increase the number of determination factors when selecting a practitioner, and more optimal patient-practitioner matching can be expected.

(3)

There is provided the matching assistance device according to the above (1) or (2), wherein the patient information includes information for setting whether to disclose the treatment history information, and only in a case where a request is received from the practitioner-side terminal to display the treatment history information associated with the patient information including information that permits the disclosure of the treatment history information, the first transmission unit transmits information for displaying the treatment history information to the practitioner-side terminal.

The above (3) allows the patient to set whether to disclose their treatment history information to the practitioners, which makes it possible to accommodate patients who are sensitive to the protection of their personal information.

(4)

There is provided a matching assistance method for assisting matching between a practitioner who performs a treatment and a patient who desires a treatment, wherein a computer is accessible to: a practitioner information database in which practitioner information for each of a plurality of the practitioners is registered; a patient information database in which patient information of the patient is registered; and a treatment history information database in which treatment history information associated with the practitioner information and the patient information of the patient who has received a treatment from the practitioner whose practitioner information is registered in the practitioner information database and, is registered, and wherein the computer is configured to execute: a matching processing step in which, when a search request for one of the practitioners matching the wishes of the patient is received from a patient-side terminal used by the patient, the computer retrieves the practitioner information, from the practitioner information database, that matches the patient information of the patient and returns information for displaying the search result to the patient-side terminal; a first transmission step in which, when a display request for the treatment history information associated with the patient information is received from a practitioner-side terminal used by the practitioner whose practitioner information is registered in the practitioner information database, the computer transmits information for displaying the treatment history information to the practitioner-side terminal; a second transmission step in which, when a display request for the treatment history information associated with the patient information of the patient is received from the patient-side terminal used by the patient, the computer transmits information for displaying the treatment history information to the patient-side terminal; and an information addition step in which, when a request to add advice information regarding treatment details included in the treatment history information is received from the practitioner-side terminal, the computer adds advice information received from the practitioner-side terminal and information of the practitioner who uses the practitioner-side terminal to the treatment history information, and registers the treatment history information in the treatment history information database, wherein the information of the practitioner who provided the advice information added to the treatment history information database in the information addition step is displayed in a format that does not identify the practitioner at the practitioner-side terminal, and is displayed in a format that identifies the practitioner at the patient-side terminal.

(5)

There is provided a non-transient recording medium on which a matching assistance program for assisting matching between a practitioner who performs a treatment and a patient who desires a treatment is recorded, wherein a computer is accessible to: a practitioner information database in which practitioner information for each of a plurality of the practitioners is registered; a patient information database in which patient information of the patient is registered; and a treatment history information database in which treatment history information associated with the practitioner information and the patient information of the patient who has received a treatment from the practitioner whose practitioner information is registered in the practitioner information database is registered, and wherein the program is configured to cause the computer to execute: a matching processing step in which, when a search request for one of the practitioners matching the desire of the patient is received from a patient-side terminal used by the patient, the computer retrieves the practitioner information, from the practitioner information database, that matches the patient information of the patient and returns information for displaying the search result to the patient-side terminal; a first transmission step in which, when a display request for the treatment history information associated with the patient information is received from a practitioner-side terminal used by the practitioner whose practitioner information is registered in the practitioner information database, the computer transmits information for displaying the treatment history information to the practitioner-side terminal; a second transmission step in which, when a display request for the treatment history information associated with the patient information of the patient is received from the patient-side terminal used by the patient, the computer transmits information for displaying the treatment history information to the patient-side terminal; and an information addition step in which, when a request to add advice information regarding treatment details included in the treatment history information is received from the practitioner-side terminal, the computer adds advice information received from the practitioner-side terminal and information of the practitioner who uses the practitioner-side terminal to the treatment history information, and registers the treatment history information in the treatment history information database, wherein the information of the practitioner who provided the advice information added to the treatment history information database in the information addition step is caused to be displayed in a format that does not identify the practitioner at the practitioner-side terminal, and displayed in a format that identifies the practitioner at the patient-side terminal.

### Advantageous Effects of Invention

The present invention can provide a matching assistance device, a matching assistance method, and a non-transient recording medium on which a matching assistance program is recorded, which assist matching between a patient and a practitioner who can perform a suitable treatment for the patient.

### BRIEF DESCRIPTION OF DRAWINGS

FIG. 1 is a diagram illustrating a schematic configuration example of a matching assistance system 100.

FIG. 2 is a schematic diagram illustrating an example of patient information stored in a patient information DB 321 of a storage unit 32 illustrated in FIG. 1.

FIG. 3 is a schematic diagram illustrating an example of practitioner information stored in a practitioner information DB 322 of the storage unit 32 illustrated in FIG. 1.

FIG. 4 is a schematic diagram illustrating an example of medical record information stored in a medical record information DB 323 of the storage unit 32 illustrated in FIG. 1.

FIG. 5 is a functional block diagram of a control unit 31 in a server 30 of the matching assistance system 100 illustrated in FIG. 1.

FIG. 6 is a sequence chart for explaining an operation example of the matching assistance system 100 illustrated in FIG. 1.

FIG. 7 is a schematic diagram illustrating an example of a search result screen displayed in step S4 of FIG. 6.

FIG. 8 is a schematic diagram illustrating an example of a request screen displayed in step S7 of FIG. 6.

FIG. 9 is a schematic diagram illustrating an example of a medical record screen displayed in step S11 of FIG. 6.

FIG. 10 is a sequence chart for explaining an example of an operation when a practitioner B adds a comment to the medical record information in the matching assistance system 100 illustrated in FIG. 1.

FIG. 11 is a schematic diagram illustrating an example of a medical record screen displayed on a display portion of a practitioner-side terminal 20 in step S34 of FIG. 10.

FIG. 12 is a schematic diagram illustrating an example of a medical record screen displayed on a display portion of a practitioner-side terminal 20 in step S34 of FIG. 10.

FIG. 13 is a schematic diagram illustrating an example of a medical record screen displayed on the display portion of the practitioner-side terminal 20 after advice is added from the medical record screen of FIG. 11.

FIG. 14 is a diagram illustrating an example of a medical record screen displayed on a patient-side terminal 10.

### DESCRIPTION OF EMBODIMENTS

### Outline of Matching Assistance System of Embodiments

An outline of an embodiment of a matching assistance system including a matching assistance device of the present invention will be described. The matching assistance system of the embodiment is assumed to be used, for example, when a practitioner working at a facility such as a hospital or an osteopathic clinic wants to perform a treatment on a patient during free time such as on holidays or after working hours, when a freelance practitioner who does not work at a facility wants to perform a treatment on a patient, or when a patient who cannot make the time to visit a facility wants to receive a treatment at home, etc., or the like.

A practitioner who wants to use the system logs in to a service website by operating a terminal and registers practitioner information with a server. Such practitioner information includes details of acquired qualifications relevant to the treatment, available dates and times, available area for treatment, area of expertise for treatment, years of experience, and contact information. A patient who wants to use the system logs in to the service website by operating his/her own terminal and registers patient information with the server. Such patient information includes the desired date and time of treatment, the desired area of treatment, the requirements of the practitioner, and personal information.

When the patient operates the terminal and requests the server to search for a practitioner that meets his/her request, the server performs a search based on the practitioner information registered in advance and the patient information registered by the patient, and the search results are displayed on the patient's terminal. The patient selects a preferred practitioner from the search results displayed on the terminal and makes a treatment request to that practitioner.

When a treatment request is made, the server sends the treatment request to the terminal of the selected practitioner, and when the practitioner performs an operation to grant the treatment request, a treatment appointment between the patient and the practitioner is established. According to this appointment, the practitioner will perform the treatment on the patient.

After performing the treatment on the patient, the practitioner logs in to the service website to create and register medical record information of the patient. The medical record information of the patient registered in this way can be viewed from the terminals of all practitioners registered in the system when the patient grants the disclosure thereof.

This system allows a practitioner who receives a treatment request to decide whether to grant the request by referring to the medical record information of the patient who made the request.

For example, a patient a wishes to be treated by a practitioner b and, by referring to past treatment details of the patient a based on the medical record information, the practitioner b may determine that patient a′s symptoms are unlikely to be resolved by practitioner b's knowledge and experience. In such a case, the practitioner b will make a determination to refuse the treatment request by patient a. In this way, the patient a can reduce the possibility of an unsuitable treatment being performed on him/her. A patient who is denied a treatment request can find a suitable practitioner for his/her symptoms by requesting a different practitioner or by changing the desired requirements and searching again.

Thus, the matching assistance system of the embodiment allows a practitioner to encounter a patient who can undergo a treatment that can be expected to improve his/her symptoms. A patient can find a practitioner who is able to perform a treatment that can be expected to improve the patient's condition. Thus, a system that benefits both practitioners and patients can be provided.

### Overall Configuration of Matching Assistance System of Embodiments

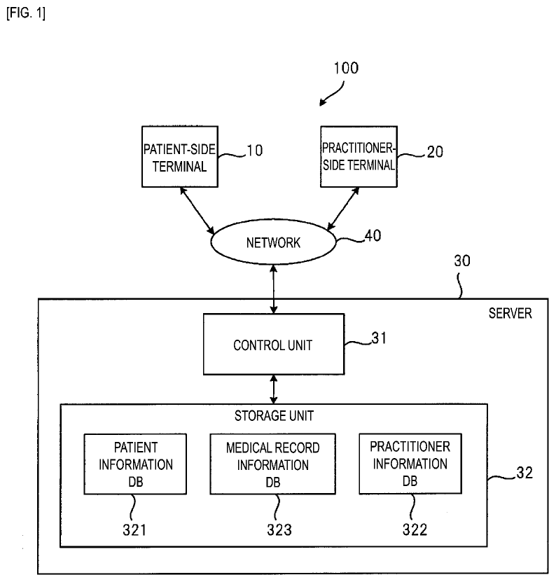

FIG. 1 is a diagram illustrating a schematic configuration example of a matching assistance system 100. As illustrated in FIG. 1, the matching assistance system 100 includes a server 30, a network 40 such as the Internet, a patient-side terminal 10 used by patients using the system to access the server 30 via the network 40, and a practitioner-side terminal 20 used by practitioners using the system to access the server 30 via the network 40.

The patient-side terminal 10 and the practitioner-side terminal 20 are electronic devices such as a smartphone, a personal computer, or a tablet-type terminal that each include display of information using a display portion, an operation interface such as a keyboard, a mouse, or a touch panel for inputting information, and a communication interface for connecting to the network 40. In FIG. 1, only one patient-side terminal 10 and one practitioner-side terminal 20 are illustrated, but in reality, all patients and practitioners using the system use a terminal.

The server 30 includes a control unit 31 and a storage unit 32 including a device capable of storing information, such as a hard disk drive or a solid state drive (SSD). The server 30 constitutes a matching assistance device.

The control unit 31 includes a central processing unit (CPU), a random access memory (RAM), and a read only memory (ROM), and executes general control of the entire system according to a program. The ROM of the control unit 31 stores programs including a matching assistance program.

The storage unit 32 stores a patient information database (DB) 321, which is a set of patient information transmitted from each patient-side terminal 10, a practitioner information DB 322, which is a set of practitioner information transmitted from each practitioner-side terminal 20, and a medical record information DB 323, which is a set of medical record information transmitted from practitioner-side terminals 20 of practitioners who performed treatments. The medical record information in the present embodiment constitutes treatment history information. In addition, the medical record information DB 323 constitutes the treatment history information database.

### Patient Information Registered in Patient Information DB

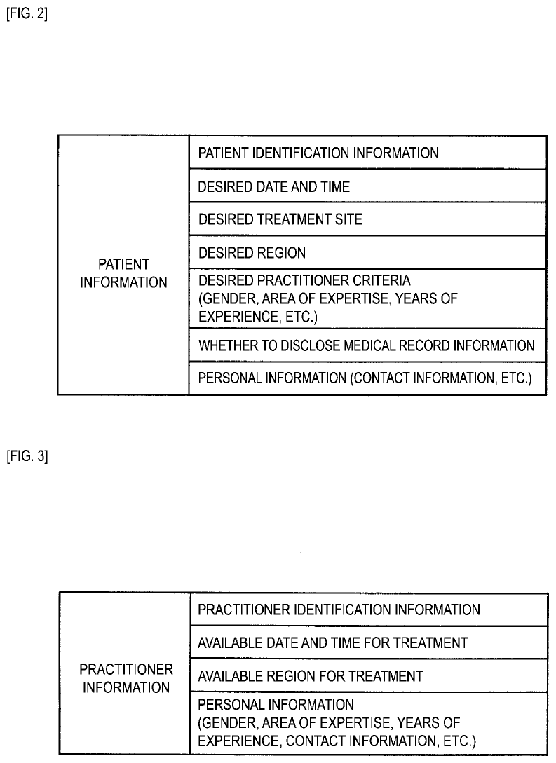

FIG. 2 is a schematic diagram illustrating an example of patient information stored in the patient information DB 321 of the storage unit 32 illustrated in FIG. 1. The patient information illustrated in FIG. 2 includes “patient identification information”, “desired date and time”, “desired treatment site”, “desired region”, “desired practitioner criteria”, “whether to disclose medical record information”, and “personal information”.

“Patient identification information” is information used to identify the patient. For example, a login ID used when the patient logs in to the service website provided by the server 30 using the patient-side terminal 10 is used as the patient identification information.

“Desired date and time” is information that indicates the date and time when the patient wants to receive the treatment.

“Desired treatment site” is information that indicates the site of the body (neck, shoulders, lower back, arms, lower body, upper body, full body, etc.) where the patient wants to receive treatment.

“Desired region” is information that indicates the region (e.g., city, town, or village) where the patient wants to receive treatment.

“Desired practitioner requirement” is information that indicates the requirement required of the practitioner, including, for example, the practitioner's gender, area of expertise (i.e., treatment site of specialization such as lower back, knees, shoulders, etc.), and years of experience.

“Whether to disclose medical record information” is information indicating whether to disclose medical record information to the practitioner, and is either “refuse disclosure” or “permit disclosure”.

“Personal information” is basic personal information needed to receive treatment, such as the patient's name, address, phone number, email address, and payment method information.

The patient information illustrated in FIG. 2 is registered in the patient information DB 321 under the control of the control unit 31 when the patient accesses the server 30 using the patient-side terminal 10, logs in to the service website, and inputs information by operating the operation interface.

### Practitioner Information Registered in Practitioner Information DB

FIG. 3 is a schematic diagram illustrating an example of practitioner information stored in the practitioner information DB 322 of the storage unit 32 illustrated in FIG. 1. The practitioner information illustrated in FIG. 3 includes “practitioner identification information”, “available date and time for treatment”, “available region for treatment”, and “personal information”.

“Practitioner identification information” is information used to identify the practitioner. For example, the login ID used when the practitioner logs in to the service site provided by the server 30 using the practitioner-side terminal 20 is used as the practitioner identification information.

“Available date and time for treatment” is information that indicates the date and time when the practitioner can perform the treatment.

“Available region for treatment” is information that indicates the region (e.g., city, town, and village) where the practitioner can perform the treatment.

“Personal information” includes the practitioner's gender, area of expertise, years of experience, qualifications held, and contact information.

The practitioner information illustrated in FIG. 3 is registered in the practitioner information DB 322 under the control of the control unit 31 when the practitioner accesses the server 30 using the practitioner-side terminal 20, logs in to the service website, and inputs information by operating the operation interface.

### Medical Record Information Registered in Medical Record Information DB

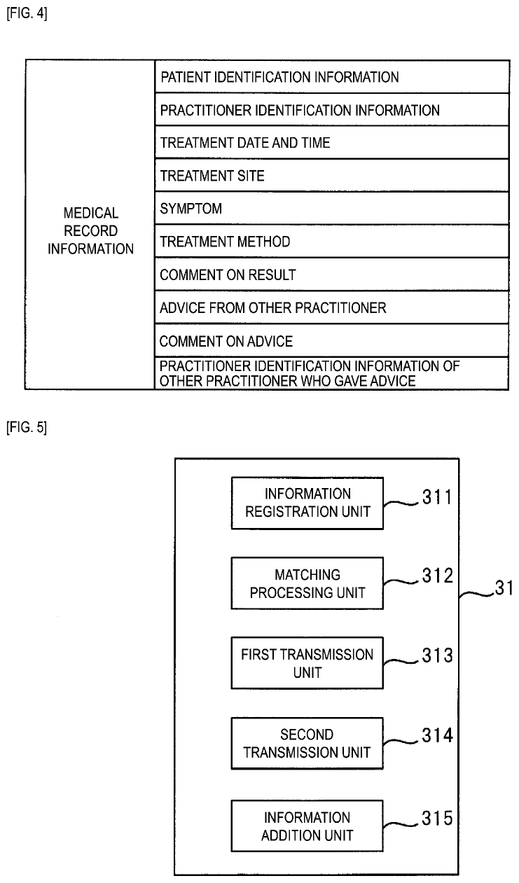

FIG. 4 is a schematic diagram illustrating an example of medical record information stored in the medical record information DB 323 of the storage unit 32 illustrated in FIG. 1. The medical record information illustrated in FIG. 4 includes “patient identification information”, “practitioner identification information”, “treatment date and time”, “treatment site”, “symptom”, “treatment method”, “comment on result”, “advice from other practitioner”, “comment on advice”, and “practitioner identification information of other practitioner who gave advice”.

“Patient identification information” is the patient identification information included in the patient information of the patient who received the treatment.

“Practitioner identification information” is the practitioner identification information included in the practitioner information of the practitioner who performed the treatment.

The “treatment date and time” is information that indicates the date and time when the treatment was performed.

The “treatment site” is information that indicates the site of the body where the treatment was performed.

“Symptom” is information that indicates the state of the patient's physical disorder such as pain, and is determined by the practitioner through a medical interview or the like.

“Treatment method” is information that indicates the details of the treatment performed (manual, physical therapy (low frequency), etc.).

“Comment on result” is information that indicates how the symptoms changed as a result of the treatment.

“Advice from other practitioner” and “practitioner identification information of other practitioner who gave advice” are information that is added to the medical record information only when there is an additional request from another practitioner different to the practitioner (i.e., the practitioner identified by the practitioner identification information in the medical record information) who created the medical record information, which will be described in detail below.

More specifically, the “advice from other practitioner” is information that indicates the content of advice (in other words, suggestions) for the “treatment method” included in this medical record information. “Practitioner identification information of other practitioner who gave advice” is the practitioner identification information included in the practitioner information of the other practitioner above who made the additional request.

“Comment on advice” is information that is added to the medical record information only when “advice from other practitioner” information has been added to the medical record information and the practitioner who created the medical record information requests to add an opinion comment for the advice, which will be described in detail below. More specifically, “comment on advice” is information that indicates the practitioner's opinion, such as a positive or negative opinion on the content of the “advice from other practitioner.

The medical record information illustrated in FIG. 4 is registered in the medical record information DB 323 under the control of the control unit 31 when a practitioner who has performed a treatment on a patient accesses the server 30 using the practitioner-side terminal 20, logs in to the service website, and inputs information by operating the operation interface.

As illustrated in FIG. 4, the medical record information includes the patient identification information of the patient who received the treatment and the practitioner identification information of the practitioner who performed the treatment. In other words, the medical record information stored in the medical record information DB 323 is associated with the patient information in the patient information DB 321 including the patient identification information included in this medical record information, and the practitioner information in the practitioner information DB 322 including the practitioner identification information contained in this medical record information.

When more than one practitioner performs a treatment on a single patient, medical record information including the practitioner identification information of each practitioner is generated and registered in the medical record information DB 323.

### Function of Control Unit in Server

FIG. 5 is a functional block diagram of a control unit 31 in a server 30 of the matching assistance system 100 illustrated in FIG. 1. The processor of the control unit 31 functions as an information registration unit 311, a matching processing unit 312, a first transmission unit 313, a second transmission unit 314, and an information addition unit 315 by executing the above-described matching assistance program stored in the ROM.

The information registration unit 311 registers patient information by storing the patient information input by the patient operating the patient-side terminal 10 in the storage area of the patient information DB 321 in the storage unit 32. In addition, the information registration unit 311 registers practitioner information by storing the practitioner information input by the practitioner operating the practitioner-side terminal 20 in the storage area of the practitioner information DB 322 in the storage unit 32. In addition, the information registration unit 311 registers medical record information by storing the medical record information input by the practitioner operating the practitioner-side terminal 20 in the storage area of the medical record information DB 323 in the storage unit 32.

The matching processing unit 312, upon receiving a request from the patient-side terminal 10 (i.e., the patient-side terminal 10 currently logged in to the service website) used by the patient identified by the patient identification information of any patient information registered in the patient information DB 321 to search for a practitioner who matches the patient's wishes, searches for practitioner information matching the patient information from the practitioner information DB 322 and returns, via the network 40, information for displaying the search results to the patient-side terminal 10 that made the search request.

The method of searching for the practitioner information that matches the patient information from among multiple practitioner information is not limited. For example, in the practitioner information, the available date and time for treatment, the available region for treatment, and the gender, area of expertise, and years of experience in the personal information, which satisfy all of the criteria of the desired date and time, the desired region, and the desired practitioner criteria in the patient information, need only be extracted as the practitioner information that matches the patient information.

When the first transmission unit 313 receives, from the practitioner-side terminal 20 (that is, the practitioner-side terminal 20 that is currently logged in to the service website) used by the practitioner whose practitioner information is registered in the practitioner information DB 322, a request for displaying medical record information associated with any patient information, for which “allow disclosure” is registered as information of “whether to disclose medical record information”, registered in the patient information DB 321 among the patient information registered in the patient information DB 321, the information for displaying this medical record information is transmitted to the practitioner-side terminal 20 that made the display request.

In other words, the first transmission unit 313, upon receiving a request from the practitioner-side terminal 20 of the practitioner who registers with the system to view the medical record information of the patient who has set to permit disclosure of the medical record information, transmits information to that practitioner-side terminal 20 to display the medical record information for which this viewing request has been made. The function of the first transmission unit 313 may allow all practitioners registered in the system to view the medical record information of all patients who have set to permit disclosure on the service website.

The second transmission unit 314, upon receiving a request from the patient-side terminal 10 (i.e., the patient-side terminal 10 currently logged in to the service website) used by the patient identified by the patient identification information of any patient information registered in the patient information DB 321 to display medical record information associated with any patient information, transmits the information for displaying the medical record information to the patient-side terminal 10 via the network 40.

In other words, the second transmission unit 314, upon receiving a request from the patient-side terminal 10 of a patient who registers with the system to view his/her own medical record information, transmits information to that patient-side terminal 10 to display the medical record information for which the viewing request has been made. The function of the second transmission unit 314 allows each patient registered in the system to view only the medical record information associated with his/her own patient information on the service website.

In addition, while a detailed explanation will be omitted, the functions of the control unit 31 allow patients who register with the system to view all practitioner information registered in the practitioner information DB 322 by logging in to the service website using the patient-side terminal 10. Similarly, a practitioner who registers with the system can log in to the service website using the practitioner-side terminal 20 to view all the patient information registered in the patient information DB 321 on the service website, with personal information withheld.

The information addition unit 315, upon receiving a request from the practitioner-side terminal 20 to add advice information regarding the “treatment details” included in the medical record information, to the medical record information that is based on the information for displaying the medical record information transmitted from the first transmission unit 313 to the practitioner-side terminal 20, adds the advice information and the practitioner identification information of the practitioner using the practitioner-side terminal 20 to the medical record information and registers this information in the medical record information DB 323.

That is, when a practitioner viewing any patient's medical record information (denoted as C1) at the practitioner-side terminal 20 (a practitioner different to the one who has created this medical record information C1) performs an operation on the service website to add an advice comment to the treatment details included in the medical record information C1, the information of the added advice comment is transmitted from the practitioner-side terminal 20 to the server 30. Then, the information of the advice comment is registered in the “advice from other practitioner” part of the medical record information C1 by the information addition unit 315 that has received the information of the advice comment.

The comment added to the medical record information C1 can be viewed by all practitioners registered in the system, as described above. In addition, only patients, among those registered with the system, identified by the patient information associated with the medical record information C1 can view this comment.

Additionally, upon receiving a request to add information of an opinion comment regarding the advice to the medical record information to which “advice from other practitioner” information has been added, from the practitioner-side terminal 20 used by the practitioner identified by the practitioner information associated with the medical record information (in other words, the practitioner who created the medical record information), the information addition unit 315 further performs a process of adding the information of an opinion comment to the medical record information and registers this information in the medical record information DB 323.

In other words, in a case where the practitioner who created the medical record information C1 is viewing the medical record information C1 at the practitioner-side terminal 20, when performing an operation on the service website to add an opinion comment regarding the “advice from other practitioner” included in the medical record information C1, the information of this added opinion comment is transmitted from the practitioner-side terminal 20 to the server 30. Then, the information of this opinion comment is registered in the “comment on advice” part of the medical record information C1 by the information addition unit 315 that has received the information of this opinion comment.

The “comment on advice” added to the medical record information C1 can be viewed by all practitioners registered in the system, as described above. In addition, only patients, among those registered with the system, identified by the patient information associated with the medical record information C1 can view this comment.

### Operation Example of Matching Assistance System

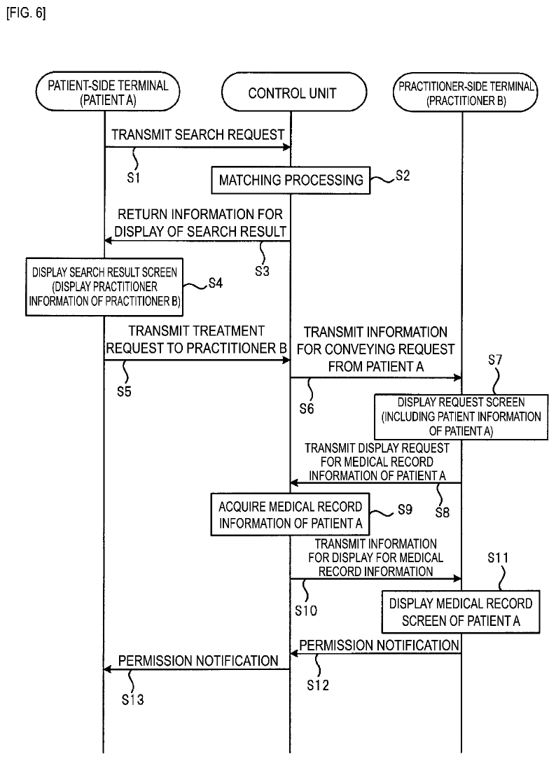

FIG. 6 is a sequence chart for explaining an operation example of the matching assistance system 100 illustrated in FIG. 1. FIG. 6 illustrates an example of how a patient A makes a search request for a practitioner that matches his/her wishes, and as a result, a practitioner B is displayed as a matching result, followed by the completion of a treatment appointment between the patient A and the practitioner B. It is assumed that the patient information of the patient A and the practitioner information of the practitioner B have already been registered in the storage unit 32.

The patient A accesses and logs in to the service website provided by the server 30 at his/her patient-side terminal 10 using a web browser or a dedicated application. Then, when the patient A operates the patient-side terminal 10 and makes a search request for practitioner information that matches his/her patient information, this search request is transmitted from the patient-side terminal 10 to the control unit 31 (step S1).

The matching processing unit 312 of the control unit 31, upon receiving the search request, performs matching processing of searching for practitioner information that is close to or meets the desired criteria included in the patient information of the patient A from among the practitioner information registered in the practitioner information DB 322 (step S2).

Then, the matching processing unit 312 generates information for displaying a list of retrieved practitioner information (information for display of search results), and returns this information for display to the patient-side terminal 10 of the patient A (step S3). Here, a case where only practitioner information of the practitioner B has been output as the practitioner information as a search result will be described.

After receiving this information for display, the patient-side terminal 10 displays a search result screen based on the information for display in the display portion by the function of the web browser or dedicated application (step S4).

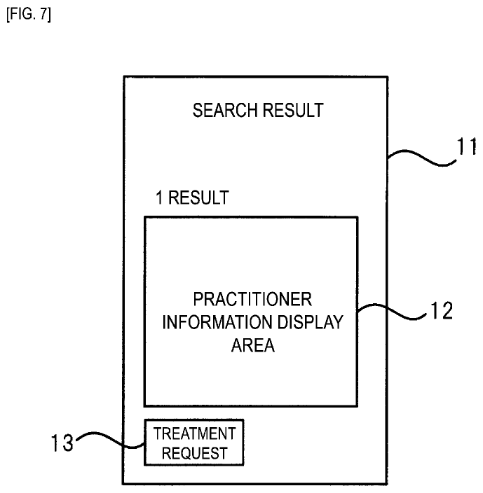

FIG. 7 is a schematic diagram illustrating an example of the search result screen displayed in the step S4 of FIG. 6. The search result screen 11 illustrated in FIG. 7 displays a comment (“1 result”) indicating the number of practitioner information results of the search, a practitioner information display area 12 where details of the practitioner information of the retrieved practitioner B are displayed, and a treatment request button 13 for making a treatment request to the practitioner B.

When the patient A, who has viewed the search result screen 11 illustrated in FIG. 7, operates the patient-side terminal 10 and presses the treatment request button 13, a treatment request to the practitioner B is transmitted from the patient-side terminal 10 to the control unit 31 (step S5).

The control unit 31, who has received this treatment request, transmits information for conveying that the treatment request has been made by the patient A to the practitioner-side terminal 20 of the practitioner B (step S6). The transmission of this information to the practitioner-side terminal 20 of practitioner B is performed based on the contact information included in the practitioner information of the practitioner B, for example, via e-mail.

The practitioner B, after confirming the e-mail at the practitioner-side terminal 20, logs in to the service website. When the practitioner B logs in to the service website, the control unit 31 transmits information to the practitioner-side terminal 20 of the practitioner B for displaying information indicating that a treatment request has been received from the patient A. The practitioner-side terminal 20 that has received this information displays a request screen based on this information in the display portion by the function of the web browser or dedicated application (step S7).

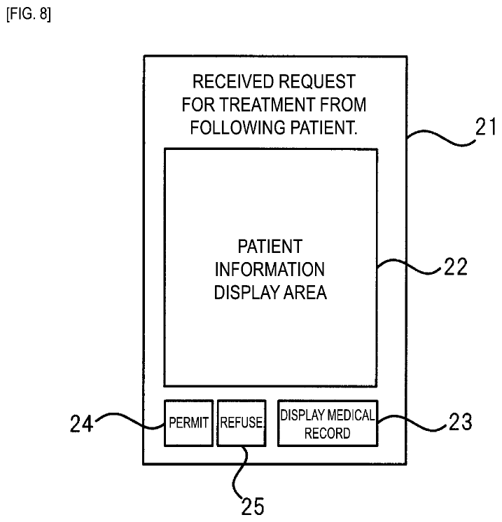

FIG. 8 is a schematic diagram of an example of the request screen displayed in step S7 of FIG. 6. The request screen 21 illustrated in FIG. 8 includes a patient information display area 22 where the details of the patient information of the patient A who made the treatment request are displayed, a medical record display button 23 for requesting the display of the medical record information associated with the patient information of the patient A, a permission button 24 for permitting the treatment request from the patient A, and a refusal button 25 for refusing the treatment request from the patient A.

When the practitioner B, who has viewed the request screen 21 illustrated in FIG. 8, operates the practitioner-side terminal 20 and presses the medical record display button 23, a request to display the medical record information of the patient A is transmitted from the practitioner-side terminal 20 to the control unit 31 (step S8).

The first transmission unit 313 of the control unit 31, which has received this display request, acquires the medical record information associated with the patient information of the patient A from the storage unit 32 (step S9), and transmits the information for display for displaying the acquired medical record information to the practitioner-side terminal 20 of the practitioner B (step S10).

The practitioner-side terminal 20 that has received this information for display displays a medical record screen based on this information for display in the display portion using the functions of the web browser or dedicated application (step S11).

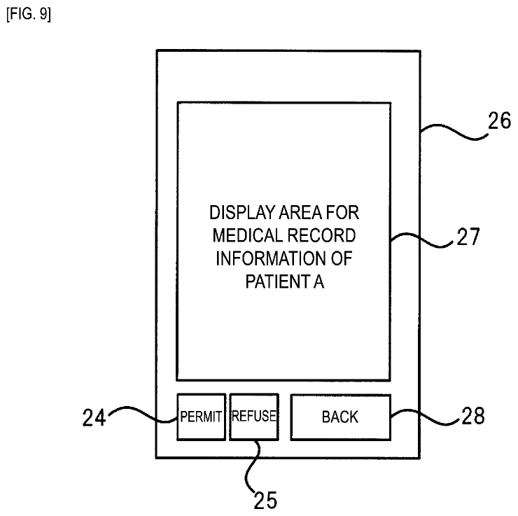

FIG. 9 is a schematic diagram illustrating an example of the medical record screen displayed in step S11 of FIG. 6. The medical record screen 26 illustrated in FIG. 9 includes a medical record information display area 27 where details of the medical record information of the patient A are displayed, a back button 28 for returning to the request screen 21 illustrated in FIG. 8, and the permission button 24 and the refusal button 25 described above.

If the practitioner B, as a result of confirming the medical record information of the patient A on the medical record screen 26, determines that his/her treatment method will be effective, the practitioner B presses the permission button 24. On the other hand, if the practitioner B, as a result of confirming the medical record information of the patient A on the medical record screen 26, determines that he/she cannot improve the patient's symptom, the practitioner B presses the refusal button 25.

When the permission button 24 is pressed, a permission notification is transmitted from the practitioner-side terminal 20 of the practitioner B to the control unit 31 (step S12). The control unit 31 that has received this permission notification transmits the permission notification to the patient-side terminal 10 of the patient A (step S13). This establishes a treatment appointment between the patient A and the practitioner B.

When the refusal button 25 is pressed on the medical record screen 26 or on the request screen 21, a refusal notification is transmitted from the practitioner-side terminal 20 of the practitioner B to the control unit 31, which receives the refusal notification and transmits the refusal notification to the patient-side terminal 10 of the patient A. As a result, a treatment appointment between the patient A and the practitioner B is not established.

The control unit 31 that has received a refusal notification from the practitioner-side terminal 20 may transmit information for displaying a screen for input of a refusal reason to the practitioner-side terminal 20. In this case, the practitioner-side terminal 20 displays this screen for input in the display portion and prompts the practitioner B to input a refusal reason. When the refusal reason is input, the practitioner-side terminal 20 transmits the information of the refusal reason to the control unit 31. Then, the control unit 31 transmits information for displaying the refusal notification and the information of the refusal reason to the patient-side terminal 10 of the patient A. This allows the patient A to know that the treatment request has been refused and to know the refusal reason.

A case is considered where, as the refusal reason, for example, the opinion of the practitioner B is that, “a physical therapist rather than a chiropractor would be more appropriate based on the medical record” or “based on the medical record, there is a problem in the knees but the knees are not in my area of expertise”. In such a case, the patient A can be given a hint to find a suitable practitioner for himself/herself based on this refusal reason. As a result, the patient can find a practitioner who is more suited to himself/herself.

### Operation Example When Practitioner Adds Comment to Medical Record Information

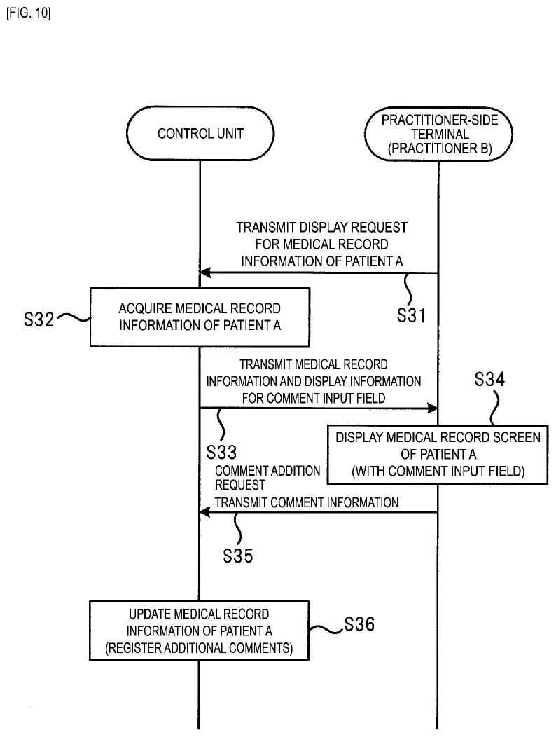

FIG. 10 is a sequence chart for explaining an example of an operation when the practitioner B adds a comment to the medical record information in the matching assistance system 100 illustrated in FIG. 1.

The practitioner B, who operates the practitioner-side terminal 20, selects the patient information of any patient A that can be viewed on the service website, and makes a request to display the medical record information associated with that patient information. Then, the display request is transmitted from the practitioner-side terminal 20 to the control unit 31 (step S31).

The first transmission unit 313 of the control unit 31, which has received this display request, acquires the medical record information associated with the patient information of the patient A from the storage unit 32 (step S32), and transmits information for display of the acquired medical record information and a comment input field to the practitioner-side terminal 20 of the practitioner B (step S33).

The comment input field in the information for display refers to a field where, when the practitioner B is the creator of the medical record information of the patient A, that is, when the practitioner identification information of the practitioner B who made the request for displaying the medical record information is included in the medical record information, this field is used to input the “opinion comment on the advice”.

On the other hand, the comment input field in the information for display refers to a field where, when the practitioner B is not the creator of the medical record information of the patient A, that is, when the practitioner identification information of the practitioner B who made the request for displaying the medical record information is not included in the medical record information, this field is used to input the “advice comment”.

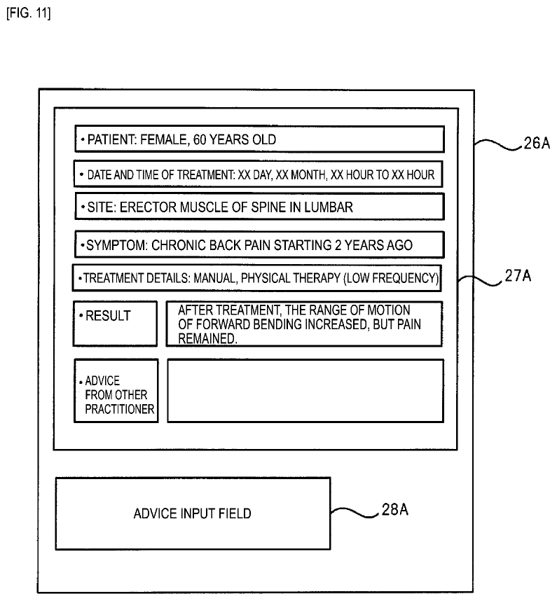

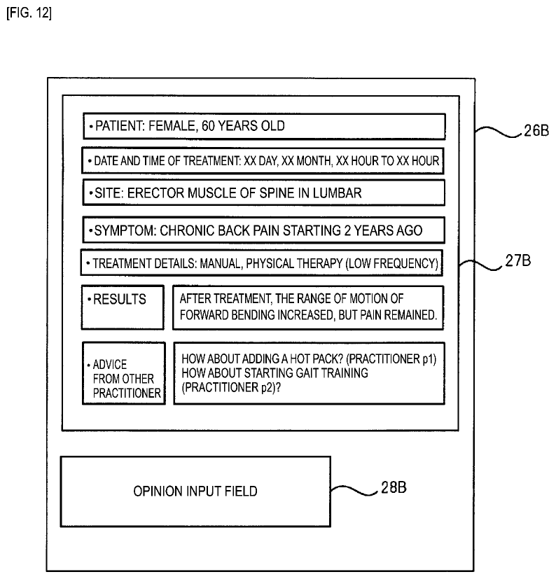

The practitioner-side terminal 20 that has received this information for display displays the medical record screen based on this information for display in the display portion using the functions of the web browser or dedicated application (step S34). FIG. 11 and FIG. 12 are schematic diagrams of an example of a medical record screen displayed in the display portion of the practitioner-side terminal 20 in the step S34 of FIG. 10.

A medical record screen 26A illustrated in FIG. 11 is an example of a screen displayed on the practitioner-side terminal 20 of the practitioner B when the practitioner B is not the creator of the medical record information of the patient A. The medical record screen 26A includes a medical record information display area 27A where details of the medical record information of the patient A are displayed, and an advice input field 28A for inputting an advice comment for the “treatment details” included in this medical record information.

A medical record screen 26B illustrated in FIG. 12 is an example of a screen displayed on the practitioner-side terminal 20 of the practitioner B when the practitioner B is the creator of the medical record information of the patient A. The medical record screen 26B includes a medical record information display area 27B where details of the medical record information of the patient A are displayed, and an opinion input field 28B for inputting an opinion comment on the “advice from other practitioner” included in this medical record information.

### Flow After Step S34 in FIG. 10 When Practitioner B is Not Creator of Medical Record Information of Patient A

When the practitioner B, as a result of confirming the medical record information of the patient A on the medical record screen 26A illustrated in FIG. 11 and determining that his/her treatment method will be effective, inputs an advice comment in the advice input field 28A and performs a confirming operation. With this confirming operation, a comment addition request to add the inputted advice comment to the medical record information of the patient A is transmitted from the practitioner-side terminal 20 to the control unit 31 along with the information of the inputted advice comment (step S35).

The information addition unit 315 of the control unit 31, which has received this comment addition request, associates the information of the comment received from the practitioner-side terminal 20 of the practitioner B with the practitioner identification information of the practitioner B, and then adds these two pieces of information to the medical record information associated with the patient information of the patient A to update the medical record information (step S36).

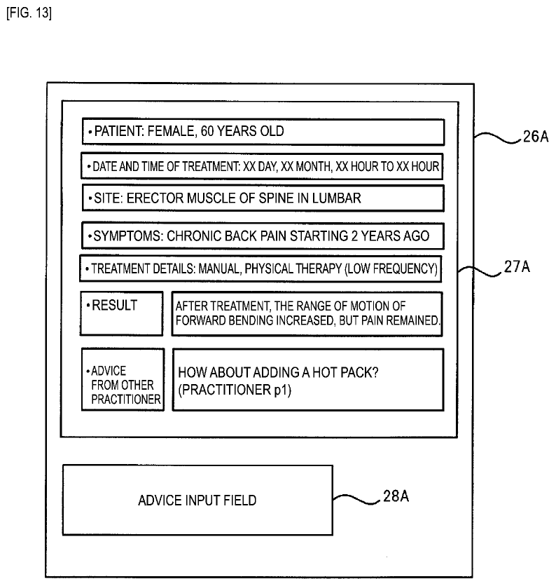

In this case, the medical record screen 26A displayed on the practitioner-side terminal 20 changes from the one illustrated in FIG. 11 to the one illustrated in FIG. 13. In FIG. 13, a comment inputted by the practitioner B (“How about adding a hot pack?”) is added to the “advice from other practitioner” field. Next to the comment, the information of the practitioner B who has made the comment is displayed in a format that does not identify the practitioner B. In the example in FIG. 12, the information is displayed in the form of a nickname such as “practitioner p1”. The words “practitioner p1” are displayed based on the practitioner identification information associated with this comment in the medical record information. For example, information with a part of the practitioner identification information deleted is displayed as a nickname.

### Flow After Step S34 in FIG. 10 When Practitioner B is Creator of Medical Record Information of Patient A

When the practitioner B, as a result of confirming the medical record information of the patient A on the medical record screen 26B illustrated in FIG. 12, wants to express an opinion, such as an opinion agreeing with or rebutting the advice from other practitioner, the practitioner B inputs an opinion comment in the opinion input field 28B and performs a confirming operation. With this confirming operation, a comment addition request to add the inputted opinion comment to the medical record information of patient A is transmitted from the practitioner-side terminal 20 to the control unit 31 along with the information of the inputted opinion comment (step S35).

The information addition unit 315 of the control unit 31, which has received this comment addition request, associates the information of the comment received from the practitioner-side terminal 20 of the practitioner B with the practitioner identification information of the practitioner B, and then adds these two pieces of information to the medical record information associated with the patient information of the patient A to update the medical record information (step S36).

### Operation When Patient A Views Medical Record Information

Here, a case where the advice information and the opinion information are added to the medical record information in the above-described flow will be described as an example.

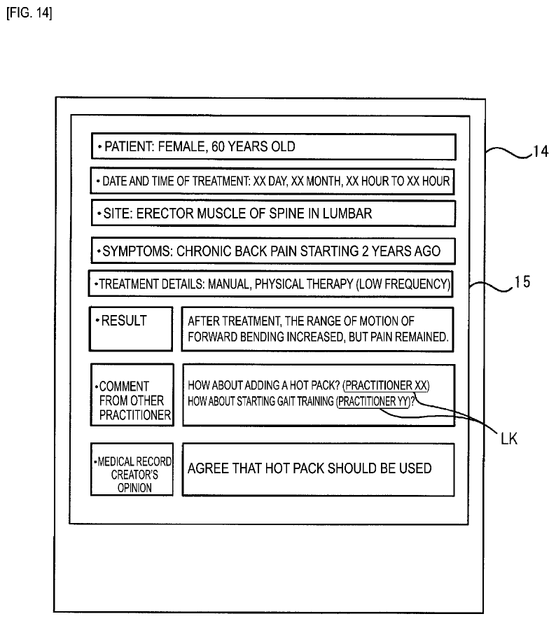

When the patient A operates the patient-side terminal 10 to request the display of his/her medical record information, the second transmission unit 314 of the control unit 31 transmits, to the patient-side terminal 10, the information for displaying the medical record screen including the medical record information of the patient A. Then, the medical record screen 14 including the medical record information of the patient A is displayed in the display portion of the patient-side terminal 10, as illustrated in FIG. 14.

The medical record screen 14 illustrated in FIG. 14 includes a medical record information display area 15 in which the medical record information of the patient A is displayed. In the medical record information display area 15, the field “comment from other practitioner” displays an advice comment from another practitioner different to the practitioner who performed the treatment on patient A, based on the information of “advice from other practitioner” in the medical record information of the patient A. Next to this advice comment, the information of the practitioner who made the comment is displayed in a format that identifies the practitioner.

For example, the information of the practitioner who made the advice comment is displayed with a name included in the practitioner information. This name can be obtained by accessing the practitioner information of the other practitioner from the practitioner identification information of the other practitioner associated with the information of the advice comment in the medical record information.

In addition, in the medical record information display area 15, in the “medical record creator's opinion” field, the opinion comment of the practitioner who performed the treatment for the patient A is displayed based on the information of “comment on advice” in the medical record information of the patient A.

In the example illustrated in FIG. 14, the name of the other practitioner who made the advice comment is further assigned a link LK. When the patient A views an advice comment and wants to confirm the practitioner information of the practitioner who made the comment, he/she presses the link LK for that name. When the link LK is pressed, the control unit 31 reads out the practitioner information from the storage unit 32 and transmits information for displaying the practitioner information to the patient-side terminal 10. This allows the patient A to immediately confirm, in the display portion, the practitioner information of the practitioner whose comments he/she is interested in.

Effect of Matching Assistance System 100

As described above, according to the matching assistance system 100, for example, a practitioner who receives a request for treatment from a patient based on the result of the matching processing can request the display of the medical record information associated with the patient information of the patient from the practitioner-side terminal 20 to confirm the past medical record information of the patient at the practitioner-side terminal 20. This allows practitioners to increase the number of factors by which to determine whether they can provide a suitable treatment for a patient based on the patient's past treatment history, thereby enabling practitioners to encounter patients who are more suited to them. In addition, patients, by disclosing their own medical record information, can expect to be selected by a practitioner who can provide a suitable treatment for them, and can encounter a practitioner who is suitable for them.

The matching assistance system 100 also allows other practitioners, who have never performed a treatment on the patient, to add advice comments to the medical record information associated with the patient. This allows that patient to view advice comments included in his/her medical record information and consider using the other practitioner who gave the advice. Further, the patient requesting treatment from the other practitioner can expect to improve his/her symptom. In this way, the advice makes it possible to increase the number of determination factors when selecting a practitioner, and more optimal matching between the patient and the practitioner can be expected. Additionally, for practitioners, the addition of advice allows them to make effective use of their own knowledge and skills, and to promote their own knowledge. As a result, it is possible to achieve matching that is beneficial to both the patient and the practitioner.

In addition, the matching assistance system 100 allows a practitioner who performed a treatment for a patient to add an opinion comment to the medical record information associated with the patient and to which advice comments given by another practitioner have been added. This allows that patient, for example, to make a decision to request another treatment from the practitioner who created the medical record information when the opinion comment is reasonable, or to make a decision to request treatment from the practitioner who gave the advice when the opinion comment is unreasonable. In this way, opinion comments can increase the number of determination factors when selecting a practitioner, and more optimal patient-practitioner matching can be expected.

The “practitioner identification information of other practitioner who gave advice” included in the medical record information is displayed in a format that does not identify the practitioner identified by the practitioner identification information (such as displaying a nickname) on the practitioner-side terminal 20, and is displayed in a format that identifies the practitioner (such as displaying a name) on the patient-side terminal 10. This gives the patient a sense of security by knowing the name of the practitioner who gave the advice.

In addition, the name of the practitioner is displayed to prevent people from writing comments for mischievous purposes. On the other hand, a practitioner who has given advice to another practitioner can prevent other practitioners from knowing that fact, thereby preventing problems between practitioners.

In the above description, it is assumed that the patient information DB 321, the practitioner information DB 322, and the medical record information DB 323 are stored in the storage unit 32 of the server 30, but the present invention is not limited to this configuration. The storage unit 32 need only be provided in a place accessible by the control unit 31 of the server 30, and may be provided in another server connected to the network 40, for example.

Although various embodiments have been described above with reference to the drawings, it goes without saying that the present invention is not limited to such examples. It will be apparent to those skilled in the art that various changes and modifications can be made within the scope of the claims, and it is understood that they also naturally fall within the technical scope of the present invention. Further, each of the components of the above-described embodiments may be combined as desired within a range that does not depart from the spirit of the present invention.

This application is based on Japanese patent application (JP 2019-039689) filed on Mar. 5, 2019, the contents of which are incorporated herein by reference.

### REFERENCE SIGNS LIST

100 Matching assistance system
10 Patient-side terminal
11 Search result screen
12 Practitioner information display area
13 Treatment request button
14 Medical record screen
15 Medical record information display area
LK Link
20 Practitioner-side terminal
21 Request screen
22 Patient information display area
23 Medical record display button
24 Permission button
25 Refusal button
26, 26A, 26B Medical record screen
27, 27A Medical record information display area
28 Back button
28A Advice input field
28B Opinion input field
30 Server
31 Control unit
311 Information registration unit
312 Matching processing unit
313 First transmission unit
314 Second transmission unit
315 Information addition unit
32 Storage unit
321 Patient information DB
322 Practitioner information DB
323 Medical record information DB
40 Network

1. A matching assistance device that assists matching between a practitioner who performs a treatment and a patient who desires a treatment,
the device comprising:
a practitioner information database in which practitioner information for each of a plurality of the practitioners is registered;
a patient information database in which patient information of the patient is registered;
a treatment history information database in which treatment history information associated with the practitioner information and the patient information of the patient who has received a treatment from the practitioner whose practitioner information is registered in the practitioner information database is registered;
a matching processing unit configured to, when a search request for one of the practitioners matching the wishes of the patient is received from a patient-side terminal used by the patient, retrieves the practitioner information, from the practitioner information database, that matches the patient information of the patient and return information for displaying the search result to the patient-side terminal;
a first transmission unit configured to, when a display request for the treatment history information associated with the patient information is received from a practitioner-side terminal used by the practitioner whose practitioner information is registered in the practitioner information database, transmit information for displaying the treatment history information to the practitioner-side terminal;
a second transmission unit configured to, when a display request for the treatment history information associated with the patient information of the patient is received from the patient-side terminal used by the patient, transmit information for displaying the treatment history information to the patient-side terminal; and
an information addition unit configured to, when a request to add advice information regarding treatment details included in the treatment history information is received from the practitioner-side terminal, add advice information received from the practitioner-side terminal and information of the practitioner who uses the practitioner-side terminal to the treatment history information, and register the treatment history information in the treatment history information database, wherein
the information of the practitioner who added the advice information included in the treatment history information is displayed in a format that does not identify the practitioner at the practitioner-side terminal, and is displayed in a format that identifies the practitioner at the patient-side terminal.

2. The matching assistance device according to claim 1, wherein
when a request to add, to the treatment history information to which the advice information has been added, information of an opinion regarding the advice is received from the practitioner-side terminal used by the practitioner identified by the practitioner information associated with the treatment history information, the information addition unit adds the information of the opinion received from the practitioner-side terminal to and registers the treatment history information in the treatment history information database.

3. The matching assistance device according to claim 1, wherein
the patient information includes information for setting whether to disclose the treatment history information, and
only in a case where a request is received from the practitioner-side terminal to display the treatment history information associated with the patient information including information that permits the disclosure of the treatment history information,
the first transmission unit transmits information for displaying the treatment history information to the practitioner-side terminal.

4. A matching assistance method for assisting matching between a practitioner who performs a treatment and a patient who desires treatment, wherein
a computer is accessible to:
a practitioner information database in which practitioner information for each of a plurality of the practitioners is registered;
a patient information database in which patient information of the patient is registered; and
a treatment history information database in which treatment history information associated with the practitioner information and the patient information of the patient who has received a treatment from the practitioner whose practitioner information is registered in the practitioner information database is registered, and wherein
the computer is configured to execute:
a matching processing step in which, when a search request for one of the practitioners matching the wishes of the patient is received from a patient-side terminal used by the patient, the computer retrieves the practitioner information, from the practitioner information database, that matches the patient information of the patient and returns information for displaying the search result to the patient-side terminal;
a first transmission step in which, when a display request for the treatment history information associated with the patient information is received from a practitioner-side terminal used by the practitioner whose practitioner information is registered in the practitioner information database, the computer transmits information for displaying the treatment history information to the practitioner-side terminal;
a second transmission step in which, when a display request for the treatment history information associated with the patient information of the patient is received from the patient-side terminal used by the patient, the computer transmits information for displaying the treatment history information to the patient-side terminal; and
an information addition step in which, when a request to add advice information regarding treatment details included in the treatment history information is received from the practitioner-side terminal, the computer adds advice information received from the practitioner-side terminal and information of the practitioner who uses the practitioner-side terminal to the treatment history information, and registers the treatment history information in the treatment history information database, and wherein
the information of the practitioner who provided the advice information added to the treatment history information database in the information addition step is displayed in a format that does not identify the practitioner at the practitioner-side terminal, and is displayed in a format that identifies the practitioner at the patient-side terminal.

5. A non-transient recording medium on which a matching assistance program for assisting matching between a practitioner who performs a treatment and a patient who desires a treatment is recorded, wherein
a computer is accessible to:
a practitioner information database in which practitioner information for each of a plurality of the practitioners is registered;
a patient information database in which patient information of the patient is registered; and
a treatment history information database in which treatment history information associated with the practitioner information and the patient information of the patient who has received a treatment from the practitioner whose practitioner information is registered in the practitioner information database is registered, and wherein
the program is configured to cause the computer to execute:
a matching processing step in which, when a search request for one of the practitioners matching the desire of the patient is received from a patient-side terminal used by the patient, the computer retrieves the practitioner information, from the practitioner information database, that matches the patient information of the patient and returns information for displaying the search result to the patient-side terminal;
a first transmission step in which, when a display request for the treatment history information associated with the patient information is received from a practitioner-side terminal used by the practitioner whose practitioner information is registered in the practitioner information database, the computer transmits information for displaying the treatment history information to the practitioner-side terminal;
a second transmission step in which, when a display request for the treatment history information associated with the patient information of the patient is received from the patient-side terminal used by the patient, the computer transmits information for displaying the treatment history information to the patient-side terminal; and
an information addition step in which, when a request to add advice information regarding treatment details included in the treatment history information is received from the practitioner-side terminal, the computer adds advice information received from the practitioner-side terminal and information of the practitioner who uses the practitioner-side terminal to the treatment history information, and registers the treatment history information in the treatment history information database, and wherein
the information of the practitioner who provided the advice information added to the treatment history information database in the information addition step is caused to be displayed in a format that does not identify the practitioner at the practitioner-side terminal, and displayed in a format that identifies the practitioner at the patient-side terminal.
